(19) United States
(12) Patent Application Publication
(10) Pub. No.: US 2025/0345562 A1
(43) Pub. Date: Nov. 13, 2025

(54) Systems and Methods of Shape Sensing Medical Devices with Electromagnetoresponsive Elements

(71) Applicant: Bard Access Systems, Inc., Salt Lake City, UT (US)

(72) Inventor: Shayne Messerly, Kaysville, UT (US)

(21) Appl. No.: 19/203,004

(22) Filed: May 8, 2025

Related U.S. Application Data
(60) Provisional application No. 63/645,027, filed on May 9, 2024.
(60) Provisional application No. 63/685,145, filed on Aug. 20, 2024.

(51) Int. Cl. A61M 25/01

(52) U.S. Cl. CPC A61M 25/0127; A61M 2025/0166

(57) ABSTRACT

A shape-sensing system includes electromagnetoresponsive elements along a length of an elongate medical device that respond to an external magnetic field generated by a magnetic interrogator. The magnetic interrogator transduces responses of the electromagnetoresponsive elements, thereby collecting location-dependent response data therefrom as they move through the external magnetic field. A console converts the location-dependent response data into raw 3D location data. The console can interpolate the raw 3D location data, thereby generating estimated 3D location data for one or more portions of the medical device between any two electromagnetoresponsive elements to provide plottable 3D location data. The console can further plot the plottable 3D location data on a display screen of the console in real-time as the medical device and the electromagnetoresponsive elements associated therewith move through the external magnetic field, thereby displaying a graphical representation of the medical device per its location, shape, and orientation in 3D space.

### PRIORITY

This application claims the benefit of priority to U.S. Provisional Application No. 63/645,027, filed May 9, 2024, and U.S. Provisional Application No. 63/685,145, filed Aug. 20, 2024, each of which is incorporated by reference in its entirety into this application.

### BACKGROUND

Intravascular guidance of medical devices including guidewires, catheters, and the like have often used fluoroscopic methods for guiding distal tips of such medical devices through vasculatures and determining whether the distal tips are appropriately placed in their target anatomical locations. However, the fluoroscopic methods expose patients and their attending clinicians to harmful X-ray radiation. Moreover, the patients can be exposed to potentially harmful contrast media needed for the fluoroscopic methods. For these reasons, some current medical research has turned to developing optical methods such as fiber-optic shape-sensing (“FOSS”) methods for the intravascular guidance of medical devices. However, FOSS systems including the medical devices thereof can be expensive for manufacturers and customers alike. In addition, such FOSS systems can be more delicate than desired.

Disclosed herein are systems and methods of shape sensing medical devices with electromagnetoresponsive elements that address the foregoing need.

### SUMMARY

Disclosed herein is a shape-sensing system for medical devices. The system includes, in some embodiments, a plurality of passive electromagnetoresponsive elements, a magnetic interrogator, and a console. The passive electromagnetoresponsive elements are along a length of an elongate medical device. Each electromagnetoresponsive element of the electromagnetoresponsive elements is responsive to an external magnetic field. The magnetic interrogator is configured to generate the external magnetic field. The magnetic interrogator is also configured to transduce responses of the electromagnetoresponsive elements to the external magnetic field, thereby collecting location-dependent response data from the electromagnetoresponsive elements as they move through the external magnetic field. The console includes memory and one or more processors. The console is configured to convert the location-dependent response data from the electromagnetoresponsive elements into raw three-dimensional (“3D”) location data for the electromagnetoresponsive elements. The console is also configured to optionally interpolate the raw 3D location data, thereby generating estimated 3D location data for one or more portions of the medical device between any two electromagnetoresponsive elements to provide plottable 3D location data with or without the estimated 3D location data. The console is also configured to plot the plottable 3D location data on a display screen of a console in real-time as the medical device and the electromagnetoresponsive elements associated therewith move through the external magnetic field, thereby displaying a graphical representation of the medical device in accordance with its location, shape, and orientation in 3D space.

In some embodiments, the medical device is a needle, a dilator, an introducer, a catheter, or a stylet configured for insertion into another elongate medical device such as another catheter.

Also disclosed herein is a medical device for a shape-sensing system. The medical device includes, in some embodiments, a plurality of passive electromagnetoresponsive elements along a length of the medical device. Each electromagnetoresponsive element of the electromagnetoresponsive elements is responsive to an external magnetic field.

In some embodiments, the medical device is a needle, a dilator, an introducer, a catheter, or a stylet configured for insertion into another medical device such as another catheter.

Also disclosed herein is a method of using a shape-sensing system. The method includes, in some embodiments, allowing a console of the shape-sensing system to automatically instantiate one or more shape-sensing processes of the console for shape-sensing with an elongate medical device. The method also includes advancing the medical device through a vasculature of a patient. The medical device has a plurality of passive electromagnetoresponsive elements along a length of the medical device. The one-or-more shape-sensing processes of the console includes generating an external magnetic field with a magnetic interrogator. The one-or-more shape-sensing processes of the console also includes transducing responses of the electromagnetoresponsive elements to the external magnetic field, thereby collecting location-dependent response data from the electromagnetoresponsive elements as they move through the external magnetic field with the advancing of the medical device. The one-or-more shape-sensing processes of the console also includes converting the location-dependent response data from the electromagnetoresponsive elements into raw 3D location data for the electromagnetoresponsive elements. The one-or-more shape-sensing processes of the console also includes optionally interpolating the raw 3D location data, thereby generating estimated 3D location data for one or more portions of the medical device between any two electromagnetoresponsive elements to provide plottable 3D location data with or without the estimated 3D location data. The one-or-more shape-sensing processes of the console also includes plotting the plottable 3D location data on a display screen of a console in real-time as the medical device and the electromagnetoresponsive elements associated therewith move through the external magnetic field, thereby displaying a graphical representation of the medical device in accordance with its location, shape, and orientation in the vasculature of the patient.

In some embodiments, allowing the console of the shape-sensing system to automatically instantiate one or more shape-sensing processes includes powering the console, selecting one or more shape-sensing modes of the console, or both.

In some embodiments, the medical device is a dilator, an introducer, a catheter, or a stylet configured for insertion into another elongate medical device such as another catheter.

In some embodiments, the medical device is a stylet.

In some embodiments, the method further includes loading the stylet into a dilator, an introducer, or a catheter.

In some embodiments, the method further includes ceasing to advance the medical device through the vasculature of the patient upon reaching a target anatomical location as determined by the shape-sensing of the medical device.

Also disclosed herein is a method of a shape-sensing system. The method includes, in some embodiments, instantiating one or more shape-sensing processes of a console for shape-sensing with an elongate medical device. The medical device has a plurality of passive electromagnetoresponsive elements along a length of the medical device. The method also includes generating an external magnetic field with a magnetic interrogator in accordance with the one-or-more shape-sensing processes. The method also includes transducing responses of the electromagnetoresponsive elements to the external magnetic field in accordance with the one-or-more shape-sensing processes, thereby collecting location-dependent response data from the electromagnetoresponsive elements as they move through the external magnetic field with the advancing of the medical device. The method also includes converting the location-dependent response data from the electromagnetoresponsive elements into raw 3D location data for the electromagnetoresponsive elements in accordance with the one-or-more shape-sensing processes. The method also includes optionally interpolating the raw 3D location data in accordance with the one-or-more shape-sensing processes, thereby generating estimated 3D location data for one or more portions of the medical device between any two electromagnetoresponsive elements to provide plottable 3D location data with or without the estimated 3D location data. The method also includes plotting, in accordance with the one-or-more shape-sensing processes, the plottable 3D location data on a display screen of the console in real-time as the medical device and the electromagnetoresponsive elements associated therewith move through the external magnetic field, thereby displaying a graphical representation of the medical device in accordance with its location, shape, and orientation in 3D space.

Also disclosed herein is a method for determining a tip of an elongate medical device is located within a superior vena cava (“SVC”). The method includes, in some embodiments, advancing the tip of the medical device through a vasculature of a patient toward the SVC. The medical device includes a plurality of passive electromagnetoresponsive elements along at least a distal-end portion of the medical device. Each electromagnetoresponsive element of the electromagnetoresponsive elements is responsive to an external magnetic field for shape sensing with a shape-sensing system including the medical device. The method also includes allowing the shape-sensing system to generate the external magnetic field with a magnetic interrogator thereof while advancing the tip of the medical device through the vasculature of the patient. The method also includes allowing the shape-sensing system to transduce responses of the electromagnetoresponsive elements to the external magnetic field, thereby collecting location-dependent response data from the electromagnetoresponsive elements as they move through the external magnetic field. The method also includes identifying on a display screen of the shape-sensing system a distinctive change in a plotted curvature of the medical device over time for a selection of the electromagnetoresponsive elements in the distal-end portion of the medical device at a moment the tip of the medical device is advanced into the SVC, thereby determining the tip of the medical device is located within the SVC.

In some embodiments, the method further includes allowing the shape-sensing system to convert the location-dependent response data from the electromagnetoresponsive elements into at least the plotted curvature of the medical device over time for displaying on the display screen.

In some embodiments, the plotted curvature of the medical device over time includes a plot of curvature vs. time for each electromagnetoresponsive element of the electromagnetoresponsive elements of the medical device.

In some embodiments, the distinctive change in the plotted curvature of the medical device over time is an instantaneous increase in the plotted curvature of the medical device over time followed by an instantaneous decrease in the plotted curvature of the medical device over time.

In some embodiments, a magnitude of the instantaneous decrease in the plotted curvature of the medical device over time is about twice that of the instantaneous increase in the plotted curvature of the medical device over time.

In some embodiments, the selection of the electromagnetoresponsive elements is a last three electromagnetoresponsive elements in the distal-end portion of the medical device.

In some embodiments, the method further includes ceasing to advance the tip of the medical device through the vasculature of the patient after determining the tip of the medical device is located in the SVC. The method also includes confirming the tip of the medical device is in the SV C by way of periodic changes in the plotted curvature of the medical device over time for the selection of the electromagnetoresponsive elements. The periodic changes in the plotted curvature of the medical device over time result from periodic changes in blood flow within the SVC as a heart of the patient beats.

In some embodiments, advancing the tip of the medical device through the vasculature of the patient includes advancing the tip of the medical device through a right internal jugular vein, a right brachiocephalic vein, and into the SVC.

In some embodiments, the medical device is a central venous catheter (“CVC”).

In some embodiments, advancing the tip of the medical device through the vasculature of the patient includes advancing the tip of the medical device through a right basilic vein, a right axillary vein, a right subclavian vein, a right brachiocephalic vein, and into the SVC.

In some embodiments, the medical device is a peripherally inserted central catheter (“PICC”).

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 is a block diagram of a first shape-sensing system for shape sensing with electromagnetoresponsive elements in accordance with some embodiments.

FIG. 2 is a block diagram of a second shape-sensing system for shape sensing with the electromagnetoresponsive elements in accordance with some embodiments.

FIG. 3 illustrates the second shape-sensing system with at least one of a stylet or a catheter as an elongate medical device including the electromagnetoresponsive elements in accordance with some embodiments.

FIG. 4 illustrates a detailed view of a distal portion of the medical device including the electromagnetoresponsive elements in accordance with some embodiments.

FIG. 5A illustrates the catheter including the electromagnetoresponsive elements in accordance with some embodiments.

FIG. 5B illustrates a needle including the electromagnetoresponsive elements in accordance with some embodiments.

FIG. 5C illustrates an introducer including the electromagnetoresponsive elements in accordance with some embodiments.

FIG. 5D illustrates a dilator including the electromagnetoresponsive elements in accordance with some embodiments.

FIG. 5E illustrates a tunneler including the electromagnetoresponsive elements in accordance with some embodiments.

FIG. 6 illustrates the second shape-sensing system with the stylet and catheter in use on a patient during a medical procedure, at least one of the stylet or the catheter being the medical device including the electromagnetoresponsive elements in accordance with some embodiments.

FIG. 7 illustrates a graphical representation of the medical device including the electromagnetoresponsive elements in accordance with its location, shape, and orientation in 3D space in accordance with some embodiments.

FIG. 8 provides plots of curvature vs. time for a selection of the electromagnetoresponsive elements in the distal-end portion of the medical device in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” is used to indicate a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal end portion,” a “proximal end section,” or a “proximal end length” of the medical device, respectively.

“Distal” is used to indicate a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal end portion,” a “distal end section,” or a “distal end length” of the medical device, respectively.

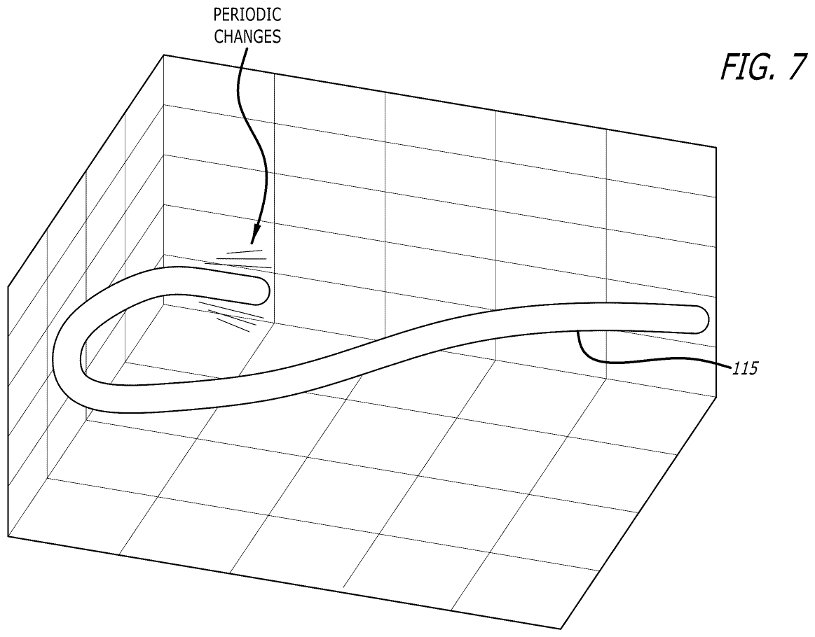

“Location” is used to indicate a location of a medical device including the electromagnetoresponsive elements in some spatial or coordinate reference system such as the magnetic interrogator-based coordinate system defined by the transducing elements of the magnetic interrogator as shown in FIG. 7 or the patient-based coordinate system set forth below. Reference points for locating the medical device in the patient-based coordinate system are provided by at least the electromagnetoresponsive elements of the medical device.

“Shape” is used to indicate a plain shape of a medical device including the electromagnetoresponsive elements in its location. By way of example, the shape of the medical device graphically represented within the magnetic interrogator-based coordinate system of FIG. 7 is a ‘J’ shape. The shape of the medical device is likewise a ‘J’ shape in the patient-based coordinate system.

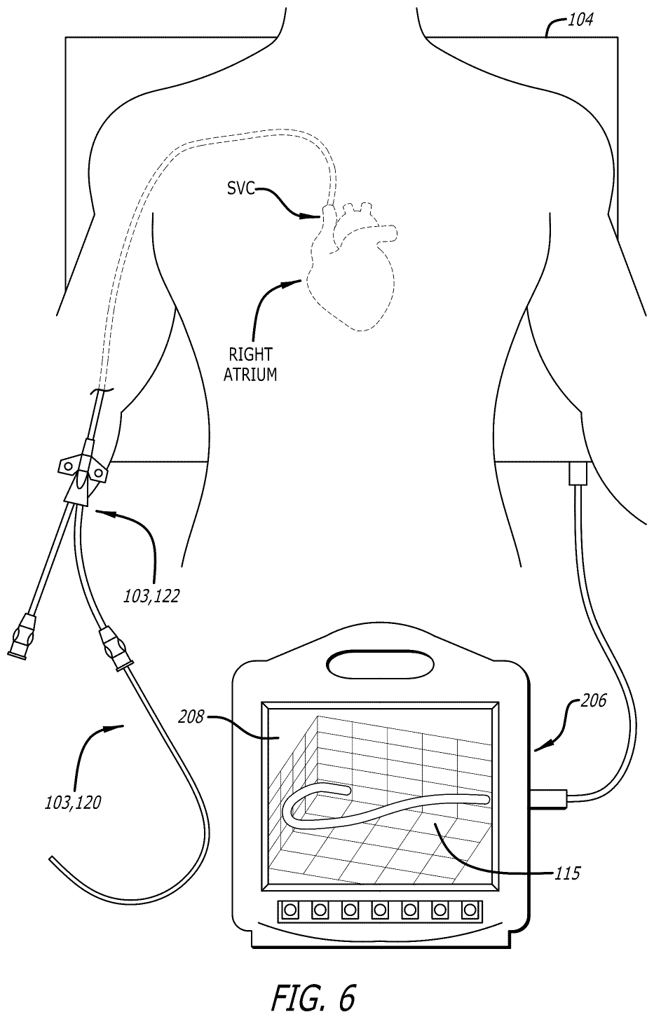

“Orientation” is used to indicate an orientation of a medical device including the electromagnetoresponsive elements in its location. By way of example, a distal tip of the medical device graphically represented within the magnetic interrogator-based coordinate system of FIG. 7 is oriented toward the yz-plane in a standard right-handed coordinate system. Upon conversion of the foregoing coordinate system to the patient-based coordinate system, the distal tip of the medical device is oriented toward the superior vena cava with an orientation toward the right atrium of the heart as shown in FIG. 6.

When used, “position” combines one or more aspects of the shape or orientation of a medical device including the electromagnetoresponsive elements in its location. By way of example, at least a distal portion of the medical device can be in malposition when the distal portion of the medical device is folded over itself such that a distal tip of the medical device is oriented away from the heart.

Unless defined otherwise, all technical and scientific terms used herein have the same meaning as commonly understood by those of ordinary skill in the art.

Again, intravascular guidance of medical devices including guidewires, catheters, and the like have often used fluoroscopic methods for guiding distal tips of such medical devices through vasculatures and determining whether the distal tips are appropriately placed in their target anatomical locations. However, the fluoroscopic methods expose patients and their attending clinicians to harmful X-ray radiation. Moreover, the patients can be exposed to potentially harmful contrast media needed for the fluoroscopic methods. For these reasons, some current medical research has turned to developing optical methods such as FOSS methods for the intravascular guidance of medical devices. However, FOSS systems including the medical devices thereof can be expensive for manufacturers and customers alike. In addition, such FOSS systems can be more delicate than desired.

Disclosed herein are systems and methods of shape sensing medical devices with electromagnetoresponsive elements that address the foregoing need.

### Shape-Sensing Systems

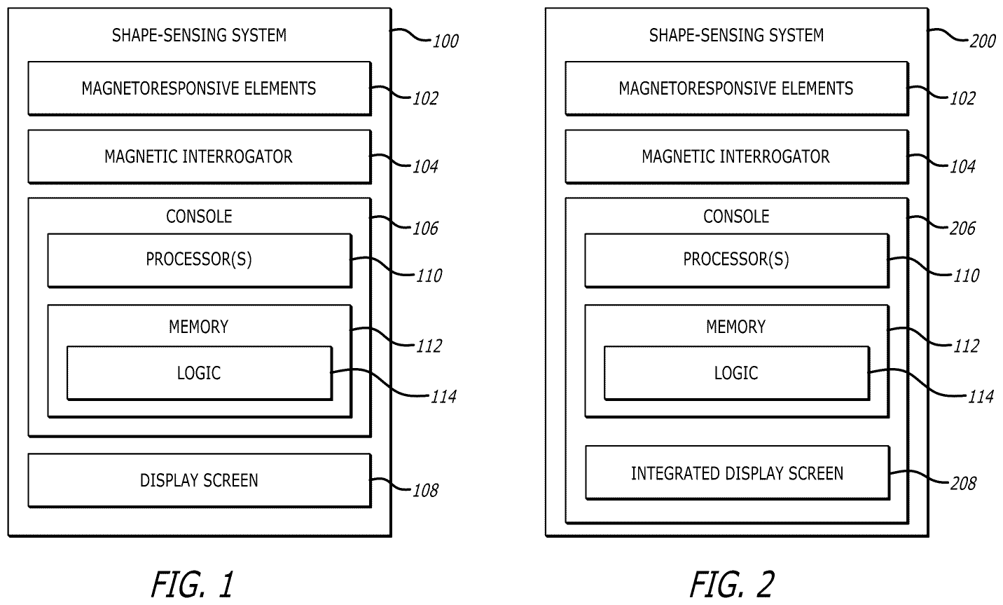

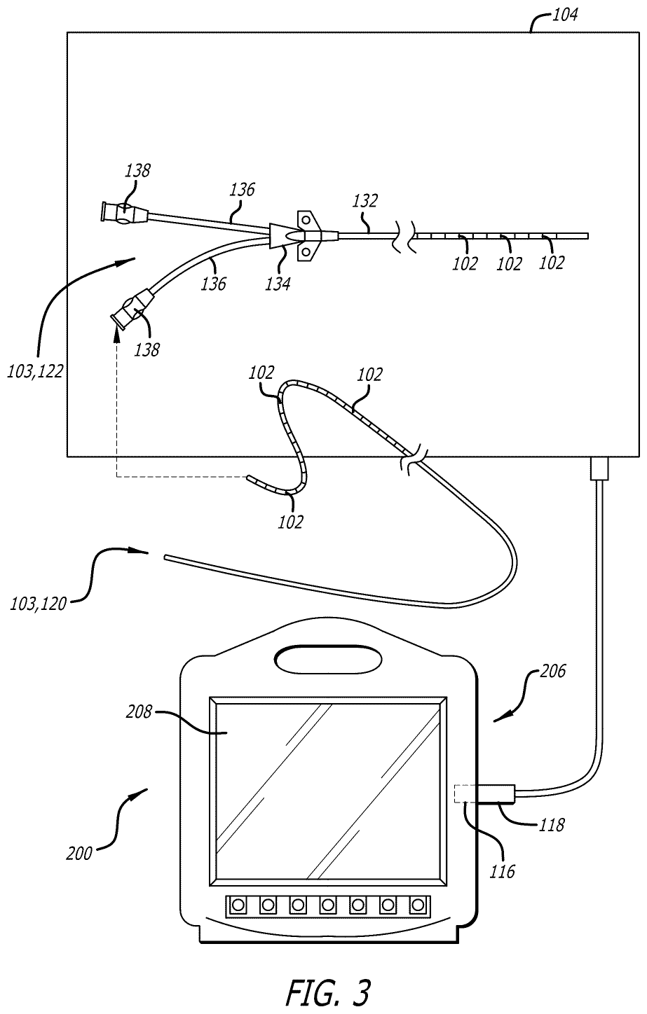

FIG. 1 is a block diagram of a first shape-sensing system 100 with a plurality of electromagnetoresponsive elements 102 in accordance with some embodiments. FIG. 2 is a block diagram of a second shape-sensing system 200 with the electromagnetoresponsive elements 102 in accordance with some embodiments. FIG. 3 illustrates the second shape-sensing system 200 with at least one of the stylet 120 or the catheter 122 as an elongate medical device 103 including the electromagnetoresponsive elements 102 in accordance with some embodiments. FIG. 6 illustrates the second shape-sensing system 200 with the stylet 120 and catheter 122 in use on a patient P during a medical procedure, at least one of the stylet 120 or the catheter 122 being the medical device 103 including the electromagnetoresponsive elements 102 in accordance with some embodiments.

As shown, the shape-sensing system 100 includes the electromagnetoresponsive elements 102, a stand-alone magnetic interrogator 104, a console 106, and a stand-alone display screen 108 such as a stand-alone monitor. The shape-sensing system 200 includes the electromagnetoresponsive elements 102, the magnetic interrogator 104, a console 206, and an integrated display screen 208, wherein the integrated display screen 208 is integrated into the console 206. However, shape-sensing systems are not limited to the shape-sensing system 100 and 200. Indeed, such shape-sensing systems are examples that convey certain concepts of shape sensing medical devices with the electromagnetoresponsive elements 102. With this in mind, description set forth below is primarily provided with respect to the shape-sensing system 200 for expository expediency, but such description can be extended to the shape-sensing system 100 and similar systems.

### Consoles

The console 206 includes one or more processors 110 and memory 112 including instructions that, when executed by the one-or-more processors 110, instantiate at least one or more shape-sensing processes for shape sensing medical devices including the electromagnetoresponsive elements 102. In addition, the console 206 includes logic 114 including at least conversion logic, interpolation logic, shaping logic, fitting logic, plotting logic, SVC-determiner logic or some combination thereof.

Configured in accordance with at least the foregoing, the console 206 converts location-dependent response data from the electromagnetoresponsive elements 102 into raw 3D location data for the electromagnetoresponsive elements 102 through a conversion process of the one-or-more shape-sensing processes utilizing the conversion logic. In addition, the console 206 optionally interpolates the raw 3D location data through an interpolation process of the one-or-more shape-sensing processes utilizing the interpolation logic, thereby generating estimated 3D location data for one or more portions of the medical device 103 between any two electromagnetoresponsive elements 102 to provide plottable 3D location data with or without the estimated 3D location data. Notably, the more electromagnetoresponsive elements 102, the more raw 3D location data to convert, which can affect frame rate if the console 206 is not adequately configured to instantaneously convert a large amount of the raw 3D location data. Interpolation of some of the raw 3D location data, such as that from every other electromagnetoresponsive element 102, can effectively mitigate frame rate issues in that interpolation is less intensive for the one-or-more processors 110. That said, if the console 206 is adequately configured to instantaneously convert a large amount of the raw 3D location data, the console 206 can generate the estimated 3D location data between any two adjacent electromagnetoresponsive elements 102 to provide finer plottable 3D location data. Lastly, through a plotting process of the one-or-more shape-sensing processes utilizing the plotting logic, the console 206 plots the plottable 3D location data on the display screen 208 in real-time as the electromagnetoresponsive elements 102 associated with some medical device 103 move through an external magnetic field of the magnetic interrogator 104, thereby displaying a graphical representation 115 of the medical device 103 in accordance with its location, shape, and orientation in 3D space as shown in FIG. 7. Being that the graphical representation 115 of the medical device 103 is in real-time, periodic changes in blood flow within an SVC while a heart of a patient beats might be noticeable in the graphical representation 115 of the medical device 103 depending upon the blood flow.

It should be understood that the graphical representation 115 of the medical device 103 shown in FIG. 7 is in accordance with its location, shape, and orientation in 3D space in a magnetic interrogator-based coordinate system defined by transducing elements (not shown) of the magnetic interrogator 104. Such a coordinate system can be converted to a patient-based coordinate system defined in a patient model established by way of one or more imaging techniques including, but not limited to, ultrasound imaging, X-ray imaging, computed tomography (“CT”) scanning, or magnetic resonance imaging (“MRI”). Alternatively, one or more physical features of a patient can be used to fit a non-specific patient model to the patient including the patient-based coordinate system. In accordance with the patient-based coordinate system, the console 206 can alternatively or additionally plot the plottable 3D location data on the display screen 208 in the plotting process to display a graphical representation 115 of the medical device 103 in accordance with its location, shape, and orientation in the vasculature of the patient like that shown in FIG. 6.

Notwithstanding the foregoing, once at least the raw 3D location data for the electromagnetoresponsive elements 102 are known, the console 206 can optionally shape such data through a shaping process of the one-or-more shape-sensing processes utilizing the shaping logic, thereby generating the shape of the medical device 103 including the electromagnetoresponsive elements 102 in accordance with the magnetic interrogator-based coordinate system. Further, the console 206 can optionally fit such a shape of the medical device 103 at any given time to some patient model and the patient-based coordinate system thereof through a fitting process of the one-or-more shape-sensing processes utilizing the fitting logic with regression analysis, thereby locating the medical device 103 by fit in the vasculature of the patient like that shown in FIG. 6.

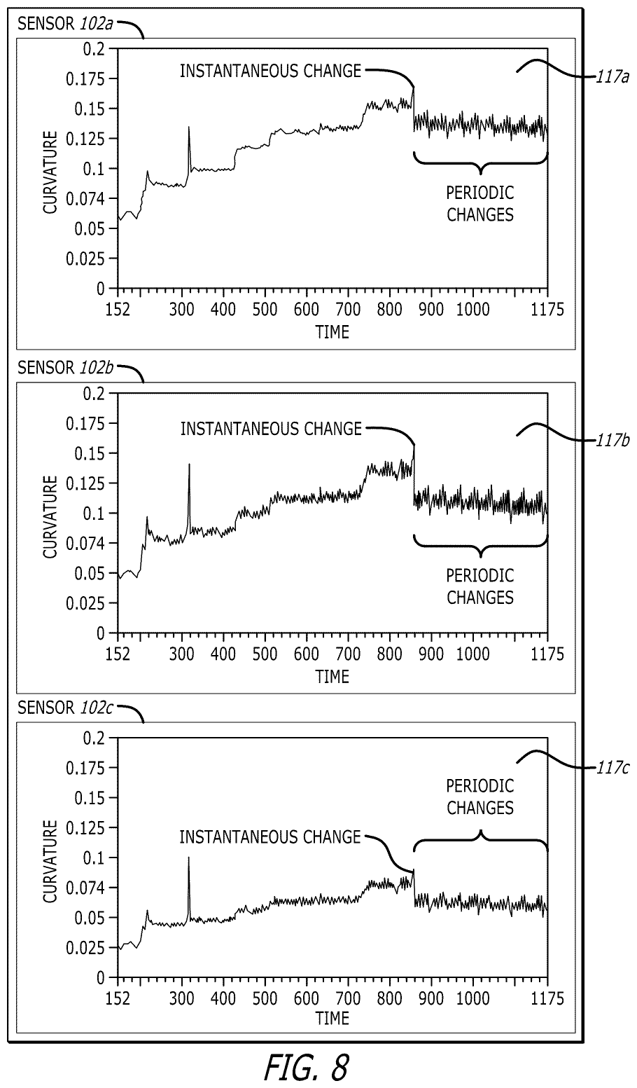

Through the plotting process of the one-or-more shape-sensing processes set forth above, the console 206 additionally or alternatively plots the curvature of the medical device 103 on the display screen 208 over time in real-time as the electromagnetoresponsive elements 102 associated with the medical device 103 move through the external magnetic field of the magnetic interrogator 104. While the console 206 can plot the curvature vs. time for each electromagnetoresponsive element 102 of the electromagnetoresponsive elements 102, FIG. 8 shows a selection of plots of curvature vs. time 117a, 117b, and 117c for a selection of electromagnetoresponsive elements 102a, 102b, and 102c in the distal-end portion of the medical device 103. The distalmost electromagnetoresponsive elements 102 such as the three electromagnetoresponsive elements 102a, 102b, and 102c in the distal-end portion of the medical device 103 are particularly useful in identifying a distinctive change in the plotted curvature of the medical device 103 in that the foregoing electromagnetoresponsive elements 102 directly experience a physical change in curvature resulting from tensile strain and compressive strain of the medical device 103 when the tip of the medical device 103 is advanced into an SVC of a patient. The distinctive change in the plotted curvature of the medical device 103 is exemplified by an instantaneous increase in the plotted curvature followed by an instantaneous decrease in the plotted curvature having a magnitude about twice that of the instantaneous increase in the plotted curvature as shown by the reference line thereto in any plot 117a, 117b, or 117c of curvature vs. time shown in FIG. 8.

In addition to being able to use any one or more of the plots of curvature vs. time to identify the distinctive change in the strain of the medical device 103 at the moment the tip of the medical device 103 is advanced into the SVC of the patient, any one or more of the plots of curvature vs. time 117a, 117b, 117c, . . . , 117n, for the selection of the electromagnetoresponsive elements 102a, 102b, 102c, . . . , 102n in the distal-end portion of the medical device 103 can be used to confirm the tip of the medical device 103 is in the SVC by way of periodic changes in the strain of the medical device 103. The periodic changes in the strain of the medical device 103 are evidenced by periodic changes in the plotted curvature of the medical device 103 sensed by the selection of the electromagnetoresponsive elements 102a, 102b, 102c, . . . , 102n. (See the three plots of curvature vs. time 117a, 117b, and 117c for the electromagnetoresponsive elements 102a, 102b, and 102c in FIG. 8, between about 860 s and 1175 s when the distal-end portion of the medical device 103 is held in position in the SVC as shown in FIG. 6.) The periodic changes in the plotted curvature result from periodic changes in blood flow within the SV C sensed by the selection of the electromagnetoresponsive elements 102 as the heart of the patient beats.

The console 206 can further include the SVC-determiner logic set forth above to automatically determine the distinctive change in the strain of the medical device 103 by way of a distinctive change in plotted curvature of the medical device 103, or the plottable data therefor, at the moment the tip of the medical device 103 is advanced into the SVC of the patient. A gain, the distinctive change in the plotted curvature is an instantaneous increase in the plotted curvature followed by an instantaneous decrease in the plotted curvature having a magnitude about twice that of the instantaneous increase in the plotted curvature. The SVC-determiner logic can also confirm the tip of the medical device 103 is in the SVC by way of automatically determining periodic changes in the plotted curvature of the medical device 103 sensed by the selection of the electromagnetoresponsive elements 102. (See the three plots of curvature vs. time 117a, 117b, and 117c for the electromagnetoresponsive elements 102a, 102b, and 102c in FIG. 8, between about 860 s and 1175 s when the distal-end portion of the medical device 103 is held in position in the SVC as shown in FIG. 6.) The periodic changes in the plotted curvature result from periodic changes in blood flow within the SVC sensed by the selection of the electromagnetoresponsive elements 102 as the heart of the patient beats. Automatically determining the distinctive and periodic changes in the strain of the medical device 103 can be used in conjunction with manually identifying the distinctive and periodic changes in the strain of the medical device 103, as above, by way of the plots of curvature vs. time 117a, 117b, 117c, . . . , 117n, for the selection of the electromagnetoresponsive elements 102a, 102b, 102c, . . . , 102n for confirmation the tip of the medical device 103 is in the SVC of the patient.

The console 206 also includes a magnetic-interrogator connector 116. The magnetic-interrogator connector 116 can be configured as a standard electrical connector complementary to that of the magnetic interrogator 104 set forth below for operably connecting the magnetic interrogator 104 to the console 206 or disconnecting the magnetic interrogator 104 from the console 206. Advantageously, the console 206 can be configured to automatically instantiate the one-or-more shape-sensing processes for shape-sensing with the electromagnetoresponsive elements 102 when the magnetic interrogator 104 is connected to the console 206.

### Magnetic Interrogator

FIGS. 3 and 6 illustrate the magnetic interrogator 104 of the shape-sensing system 200 with in accordance with some embodiments.

While not shown, the magnetic interrogator 104 is configured to electromagnetically generate the external magnetic field through which medical devices including the electromagnetoresponsive elements 102 move. The magnetic interrogator 104 is also configured to transduce responses of the electromagnetoresponsive elements 102 to the external magnetic field, thereby collecting the location-dependent response data from the electromagnetoresponsive elements 102 as the medical devices including the electromagnetoresponsive elements 102 move through the external magnetic field.

The magnetic interrogator 104 can include a console connector 118. The console connector 118 can be configured as a standard electrical connector complementary to that of the console 206 set forth above for operably connecting the magnetic interrogator 104 to the console 206 or disconnecting the magnetic interrogator 104 from the console 206.

### Electromagnetoresponsive Elements

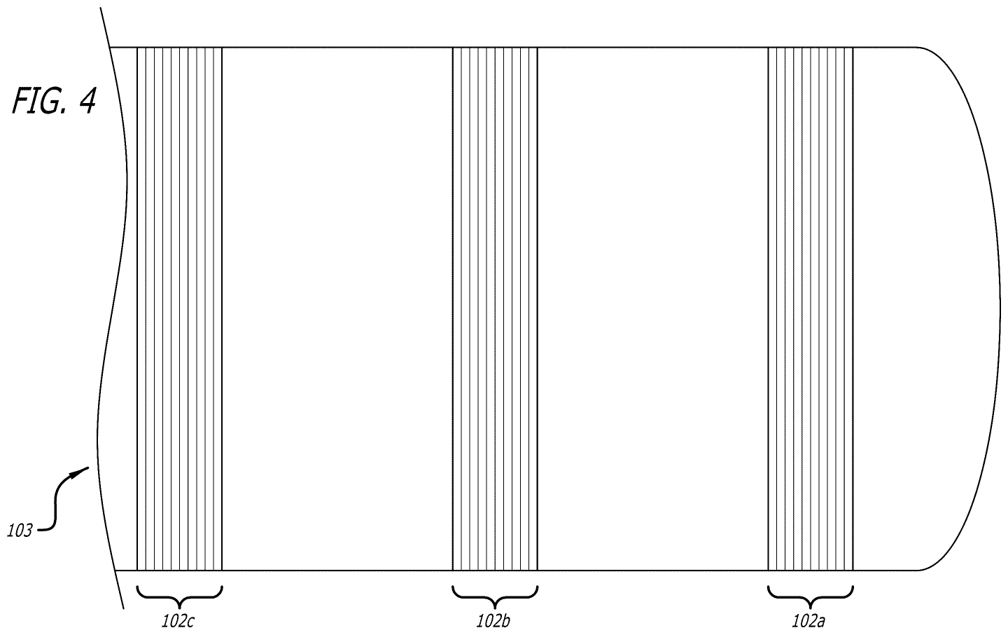

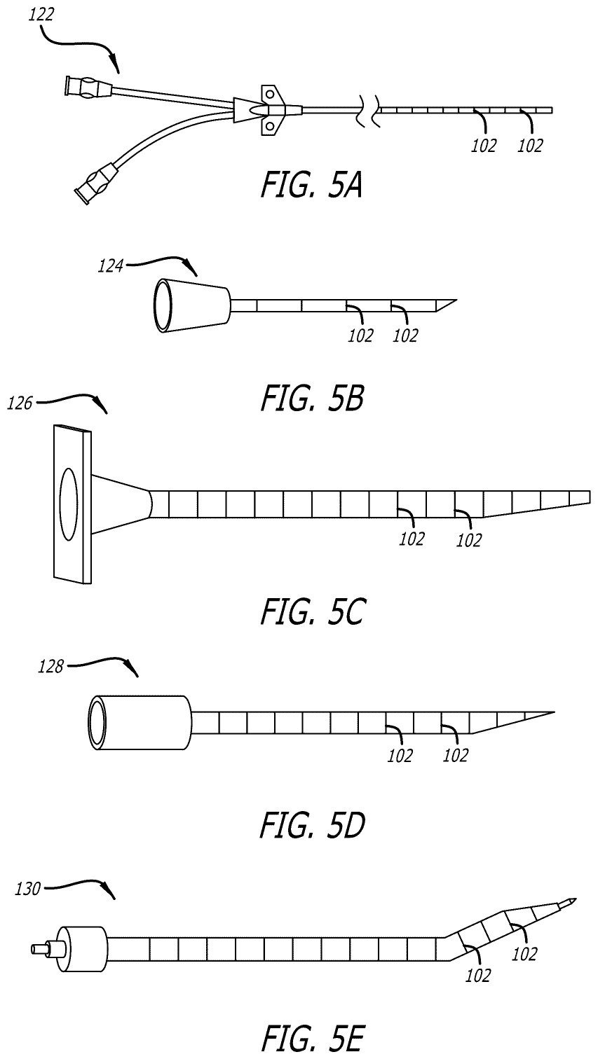

FIGS. 3 and 5A-5E illustrate the electromagnetoresponsive elements 102 included with various medical devices in accordance with some embodiments. FIG. 4 illustrates a detailed view of a distal portion of the medical device 103 including the electromagnetoresponsive elements 102 in accordance with some embodiments.

As shown, the electromagnetoresponsive elements 102 can be along at least the distal portion of the medical device 103 for shape sensing the distal portion of the medical device 103. Indeed, such electromagnetoresponsive elements are shown by way of electromagnetoresponsive elements 102a, 102b, 102c, . . . , 102n along at least the distal portion of the medical device 103 for shape sensing with the shape-sensing system 200. Notably, the electromagnetoresponsive elements 102 can be incorporated within a body of the medical device 103, such as within the catheter tube 132 of the catheter 122 set forth below, or over the body of the medical device 103, such as over the catheter tube 132 of the catheter 122, optionally, with a coating over the body of the medical device 103 to secure the electromagnetoresponsive elements 102 over the body as well as provide a smooth surface of the body of the medical device 103.

The electromagnetoresponsive elements 102 are passive electromagnetoresponsive elements in that each electromagnetoresponsive element 102 of the electromagnetoresponsive elements 102 is not internally powered by an internal power source via its corresponding medical device 103 or externally powered by an external power source operably coupled to the medical device 103. Instead, the electromagnetoresponsive elements 102 are responsive to the external magnetic field, itself. While embracing some theoretical flexibility, each electromagnetoresponsive element 102 of the electromagnetoresponsive elements 102 can have a natural frequency of vibration that, when matched by the external magnetic field of the magnetic interrogator 104, urges the electromagnetoresponsive element 102 to resonate with the external magnetic field in response. Such resonance, in turn, creates local magnetic fields about the electromagnetoresponsive elements 102 that are detected and transduced by the magnetic interrogator 104 for the location-dependent response data.

The electromagnetoresponsive elements 102 can have sufficient physical and chemical integrity for sterilization by dry heat, moist heat optionally in combination with pressure (e.g., by an autoclave), a biocide (e.g., hydrogen peroxide, ethylene oxide, etc.) optionally in combination with pressure, radiation (e.g., ultraviolet radiation), or a combination thereof when the medical device 103 including the electromagnetoresponsive elements 102 is sterilized.

### Medical Devices

FIGS. 5A-5E illustrate various medical devices into which the electromagnetoresponsive elements 102 can be incorporated in accordance with some embodiments.

The medical device 103 can be an elongate medical device such as an intravascular medical device selected from a stylet 120 or some other probe as shown in FIGS. 3 and 6, a catheter 122 such as a CVC or PICC as shown in FIG. 5A, a needle 124 as shown in FIG. 5B, an introducer 126 as shown in FIG. 5C, a dilator 128 as shown in FIG. 5D, and a tunneler 130 as shown in FIG. 5E.

With the catheter 122 as an example of the medical device 103, the catheter 122 includes a catheter tube 132, a bifurcated hub 134, two extension legs 136, and two Luer connectors 138 operably connected in the foregoing order. The catheter tube 132 includes two catheter-tube lumens, the bifurcated hub 134 has two hub lumens correspondingly fluidly connected to the two catheter-tube lumens, and each extension leg of the two extension legs 136 has an extension-leg lumen fluidly connected to a hub lumen of the two hub lumens, thereby providing two catheter lumens.

A combination of two or more of the foregoing medical devices can be loaded into one another provided at least one medical device of the combination of medical devices includes the electromagnetoresponsive elements 102. If two or more medical devices of the foregoing combination of medical devices includes the electromagnetoresponsive elements 102, the shape-sensing system 200 can advantageously utilize redundancy in shape sensing the medical devices for confirmation. In an example, the stylet 120, the catheter 122, or both the stylet 120 and catheter 122 of FIGS. 3 and 6 can include the electromagnetoresponsive elements 102.

A gain, the electromagnetoresponsive elements 102 can be incorporated along a length of the medical device 103 such as within a body of the medical device 103, for example, within the catheter tube 132 of the catheter 122, or over the body of the medical device 103, for example over the catheter tube 132 of the catheter 122, optionally, with a coating over the body of the medical device 103 to secure the electromagnetoresponsive elements 102 over the body as well as provide a smooth surface of the body of the medical device 103.

### Methods

Methods of the shape-sensing system 200 or any medical devices disclosed herein include at least methods of the shape-sensing system 200, itself, or methods of using the shape-sensing system 200 or medical devices.

In an example of a method of the shape-sensing system 200, itself, the method includes, instantiating the one-or-more shape-sensing processes of the console 206 for shape-sensing with the medical device 103.

The method also includes generating an external magnetic field with the magnetic interrogator 104 in accordance with the one-or-more shape-sensing processes.

The method also includes transducing responses of the electromagnetoresponsive elements to the external magnetic field in accordance with the one-or-more shape-sensing processes, thereby collecting location-dependent response data from the electromagnetoresponsive elements 102 as they move through the external magnetic field with the advancing of the medical device 103.

The method also includes converting the location-dependent response data from the electromagnetoresponsive elements 102 into raw 3D location data for the electromagnetoresponsive elements 102 in accordance with the one-or-more shape-sensing processes.

The method also includes optionally interpolating the raw 3D location data in accordance with the one-or-more shape-sensing processes, thereby generating estimated 3D location data for one or more portions of the medical device 103 between any two electromagnetoresponsive elements 102 to provide plottable 3D location data with or without the estimated 3D location data.

The method also includes plotting, in accordance with the one-or-more shape-sensing processes, the plottable 3D location data on the display screen 208 of the console 206 in real-time as the medical device 103 and the electromagnetoresponsive elements 102 associated therewith move through the external magnetic field, thereby displaying a graphical representation 115 of the medical device 103 in accordance with its location, shape, and orientation in 3D space.

In an example of a method of using the shape-sensing system 200, the method includes, in some embodiments, allowing the console 206 of the shape-sensing system 200 to automatically instantiate the one-or-more shape-sensing processes of the console 206 for shape-sensing with the medical device 103. Allowing the console 206 to automatically instantiate the one-or-more shape-sensing processes includes powering the console 206, selecting one or more shape-sensing modes of the console 206, or both.

When the medical device 103 is the stylet 120, the method also includes loading the stylet 120 into the dilator 128, the introducer 126, or the catheter 122.

Whether the medical device 103 is the stylet 120 or some other medical device 103 like that set forth above, the method also includes advancing the medical device 103 through a vasculature of a patient. As set forth above, the medical device 103 includes the electromagnetoresponsive elements 102 along a length of the medical device 103.

A gain, the one-or-more shape-sensing processes of the console 206 includes generating an external magnetic field with the magnetic interrogator 104. The one-or-more shape-sensing processes of the console 206 also includes transducing responses of the electromagnetoresponsive elements to the external magnetic field, thereby collecting location-dependent response data from the electromagnetoresponsive elements 102 as they move through the external magnetic field with the advancing (or withdrawing) of the medical device 103. The one-or-more shape-sensing processes of the console 206 also includes converting the location-dependent response data from the electromagnetoresponsive elements 102 into raw 3D location data for the electromagnetoresponsive elements 102. The one-or-more shape-sensing processes of the console 206 also includes optionally interpolating the raw 3D location data, thereby generating estimated 3D location data for one or more portions of the medical device 103 between any two electromagnetoresponsive elements 102 to provide plottable 3D location data with or without the estimated 3D location data. The one-or-more shape-sensing processes of the console 206 also includes plotting the plottable 3D location data on the display screen 208 of the console 206 in real-time as the medical device 103 and the electromagnetoresponsive elements 102 associated therewith move through the external magnetic field, thereby displaying a graphical representation 115 of the medical device 103 in accordance with its location, shape, and orientation in the vasculature of the patient.

The method also includes ceasing to advance the medical device 103 through the vasculature of the patient upon reaching a target anatomical location as determined by the shape-sensing of the medical device 103.

In another example of a method of using the shape-sensing system 200, the method includes, in some embodiments, determining a tip of an elongate medical device is located within an SCV of a patient by way of the shape-sensing system 200.

As above, such a method includes allowing the console 206 of the shape-sensing system 200 to automatically instantiate the one-or-more shape-sensing processes of the console 206 for shape-sensing with the medical device 103. Allowing the console 206 to automatically instantiate the one-or-more shape-sensing processes includes powering the console 206, selecting one or more shape-sensing modes of the console 206, or both.

Whether the medical device 103 is the stylet 120 or some other medical device 103 like that set forth above, the method also includes advancing the medical device 103 through a vasculature of a patient toward the SVC while the one-or-more shape-sensing processes of the console 206 generate an external magnetic field with the magnetic interrogator 104.

Notably, when the medical device 103 is a CVC, advancing the medical device 103 through the vasculature of the patient toward the SVC includes advancing the medical device 103 or the tip thereof through a right internal jugular vein, a right brachiocephalic vein, and into the SVC. When the medical device 103 is a PICC, advancing the medical device 103 through the vasculature of the patient toward the SVC includes advancing the medical device 103 or the tip thereof through a right basilic vein, a right axillary vein, a right subclavian vein, a right brachiocephalic vein, and into the SVC.

Being that the medical device 103 includes the electromagnetoresponsive elements 102 along at least the distal-end portion of the medical device 103, each electromagnetoresponsive element 102 of the electromagnetoresponsive elements 102 is responsive to the external magnetic field for shape sensing with the shape-sensing system 200. As such, the method also includes allowing the shape-sensing system 200 to transduce responses of the electromagnetoresponsive elements 102 to the external magnetic field, thereby collecting location-dependent response data from the electromagnetoresponsive elements 102 as they move through the external magnetic field.

The method also includes allowing the shape-sensing system 200 to convert the location-dependent response data from the electromagnetoresponsive elements 102 into at least a plotted curvature of the medical device 103 over time for displaying on the display screen 208. As set forth above, the plotted curvature of the medical device 103 over time includes one or more plots of curvature vs. time such as plots 117a, 117b, and 117c respectively for electromagnetoresponsive elements 102a, 102b, and 102c of the medical device 103.

The method also includes identifying on the display screen 208 of the shape-sensing system 200 a distinctive change in the plotted curvature of the medical device 103 over time for a selection of the electromagnetoresponsive elements 102 in the distal-end portion of the medical device 103 at a moment the tip of the medical device 103 is advanced into the SVC, thereby determining the tip of the medical device 103 is located within the SVC. As set forth above, the distalmost electromagnetoresponsive elements 102 such as the three electromagnetoresponsive elements 102a, 102b, and 102c in the distal-end portion of the medical device 103 are particularly useful in identifying the distinctive change in the plotted curvature of the medical device 103 in that the foregoing electromagnetoresponsive elements 102 directly experience the physical change in curvature resulting from tensile strain and compressive strain of the medical device 103 when the tip of the medical device 103 is advanced into an SVC of a patient. Further, the distinctive change in the plotted curvature of the medical device 103 is exemplified by an instantaneous increase in the plotted curvature followed by an instantaneous decrease in the plotted curvature having a magnitude about twice that of the instantaneous increase in the plotted curvature as shown by the reference line thereto in any plot 117a, 117b, or 117c of curvature vs. time shown in FIG. 8.

The method also includes ceasing to advance the tip of the medical device 103 through the vasculature of the patient after determining the tip of the medical device 103 is located in the SVC.

The method also includes confirming the tip of the medical device 103 is in the SVC by way of periodic changes in the plotted curvature of the medical device 103 over time for the selection of the electromagnetoresponsive elements 102. As set forth above, the periodic changes in the plotted curvature of the medical device 103 over time result from periodic changes in blood flow within the SVC sensed by the selection of the electromagnetoresponsive elements 102 as the heart of the patient beats.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A shape-sensing system for medical devices, comprising:
a plurality of passive electromagnetoresponsive elements along a length of an elongate medical device, each electromagnetoresponsive element of the electromagnetoresponsive elements responsive to an external magnetic field;
a magnetic interrogator configured to:
generate the external magnetic field; and
transduce responses of the electromagnetoresponsive elements to the external magnetic field, thereby collecting location-dependent response data from the electromagnetoresponsive elements as they move through the external magnetic field; and
a console including memory and one or more processors configured to:
convert the location-dependent response data from the electromagnetoresponsive elements into raw three-dimensional (“3D”) location data for the electromagnetoresponsive elements;
optionally interpolate the raw 3D location data, thereby generating estimated 3D location data for one or more portions of the medical device between any two electromagnetoresponsive elements to provide plottable 3D location data with or without the estimated 3D location data; and
plot the plottable 3D location data on a display screen of a console in real-time as the medical device and the electromagnetoresponsive elements associated therewith move through the external magnetic field, thereby displaying a graphical representation of the medical device in accordance with its location, shape, and orientation in 3D space.

2. The shape-sensing system of claim 1, wherein the medical device is a needle, a dilator, an introducer, a catheter, or a stylet configured for insertion into another elongate medical device such as another catheter.

3. A medical device for a shape-sensing system, comprising:
a plurality of passive electromagnetoresponsive elements along a length of the medical device, each electromagnetoresponsive element of the electromagnetoresponsive elements responsive to an external magnetic field.

4. The medical device of claim 3, wherein the medical device is a needle, a dilator, an introducer, a catheter, or a stylet configured for insertion into another medical device such as another catheter.

5. A method of using a shape-sensing system, comprising:
allowing a console of the shape-sensing system to automatically instantiate one or more shape-sensing processes of the console for shape-sensing with an elongate medical device; and
advancing the medical device through a vasculature of a patient, the medical device having a plurality of passive electromagnetoresponsive elements along a length of the medical device, wherein the one-or-more shape-sensing processes of the console includes:
generating an external magnetic field with a magnetic interrogator;
transducing responses of the electromagnetoresponsive elements to the external magnetic field, thereby collecting location-dependent response data from the electromagnetoresponsive elements as they move through the external magnetic field with the advancing of the medical device;
converting the location-dependent response data from the electromagnetoresponsive elements into raw three-dimensional (“3D”) location data for the electromagnetoresponsive elements;
optionally interpolating the raw 3D location data, thereby generating estimated 3D location data for one or more portions of the medical device between any two electromagnetoresponsive elements to provide plottable 3D location data with or without the estimated 3D location data; and
plotting the plottable 3D location data on a display screen of a console in real-time as the medical device and the electromagnetoresponsive elements associated therewith move through the external magnetic field, thereby displaying a graphical representation of the medical device in accordance with its location, shape, and orientation in the vasculature of the patient.

6. The method of claim 5, wherein allowing the console of the shape-sensing system to automatically instantiate one or more shape-sensing processes includes powering the console, selecting one or more shape-sensing modes of the console, or both.

7. The method of claim 5, wherein the medical device is a dilator, an introducer, a catheter, or a stylet configured for insertion into another elongate medical device such as another catheter.

8. The method of claim 5, wherein the medical device is a stylet.

9. The method of claim 8, further comprising loading the stylet into a dilator, an introducer, or a catheter.

10. The method of claim 5, further comprising ceasing to advance the medical device through the vasculature of the patient upon reaching a target anatomical location as determined by the shape-sensing of the medical device.

11. A method of a shape-sensing system, comprising:
instantiating one or more shape-sensing processes of a console for shape-sensing with an elongate medical device, the medical device having a plurality of passive electromagnetoresponsive elements along a length of the medical device;
generating an external magnetic field with a magnetic interrogator in accordance with the one-or-more shape-sensing processes;
transducing responses of the electromagnetoresponsive elements to the external magnetic field in accordance with the one-or-more shape-sensing processes, thereby collecting location-dependent response data from the electromagnetoresponsive elements as they move through the external magnetic field with the advancing of the medical device;
converting the location-dependent response data from the electromagnetoresponsive elements into raw three-dimensional (“3D”) location data for the electromagnetoresponsive elements in accordance with the one-or-more shape-sensing processes;
optionally interpolating the raw 3D location data in accordance with the one-or-more shape-sensing processes, thereby generating estimated 3D location data for one or more portions of the medical device between any two electromagnetoresponsive elements to provide plottable 3D location data with or without the estimated 3D location data; and
plotting, in accordance with the one-or-more shape-sensing processes, the plottable 3D location data on a display screen of the console in real-time as the medical device and the electromagnetoresponsive elements associated therewith move through the external magnetic field, thereby displaying a graphical representation of the medical device in accordance with its location, shape, and orientation in 3D space.

12. A method for determining a tip of an elongate medical device is located within a superior vena cava (“SVC”), comprising:
advancing the tip of the medical device through a vasculature of a patient toward the SVC, the medical device including a plurality of passive electromagnetoresponsive elements along at least a distal-end portion of the medical device, each electromagnetoresponsive element of the electromagnetoresponsive elements responsive to an external magnetic field for shape sensing with a shape-sensing system including the medical device;
allowing the shape-sensing system to generate the external magnetic field with a magnetic interrogator thereof while advancing the tip of the medical device through the vasculature of the patient;
allowing the shape-sensing system to transduce responses of the electromagnetoresponsive elements to the external magnetic field, thereby collecting location-dependent response data from the electromagnetoresponsive elements as they move through the external magnetic field; and
identifying on a display screen of the shape-sensing system a distinctive change in a plotted curvature of the medical device over time for a selection of the electromagnetoresponsive elements in the distal-end portion of the medical device at a moment the tip of the medical device is advanced into the SVC, thereby determining the tip of the medical device is located within the SVC.

13. The method of claim 12, further comprising allowing the shape-sensing system to convert the location-dependent response data from the electromagnetoresponsive elements into at least the plotted curvature of the medical device over time for displaying on the display screen.

14. The method of claim 13, wherein the plotted curvature of the medical device over time includes a plot of curvature vs. time for each electromagnetoresponsive element of the electromagnetoresponsive elements of the medical device.

15. The method of claim 12, wherein the distinctive change in the plotted curvature of the medical device over time is an instantaneous increase in the plotted curvature of the medical device over time followed by an instantaneous decrease in the plotted curvature of the medical device over time.

16. The method of claim 15, wherein a magnitude of the instantaneous decrease in the plotted curvature of the medical device over time is about twice that of the instantaneous increase in the plotted curvature of the medical device over time.

17. The method of claim 12, wherein the selection of the electromagnetoresponsive elements is a last three electromagnetoresponsive elements in the distal-end portion of the medical device.

18. The method of claim 12, further comprising:
ceasing to advance the tip of the medical device through the vasculature of the patient after determining the tip of the medical device is located in the SVC; and
confirming the tip of the medical device is in the SVC by way of periodic changes in the plotted curvature of the medical device over time for the selection of the electromagnetoresponsive elements, the periodic changes in the plotted curvature of the medical device over time resulting from periodic changes in blood flow within the SVC as a heart of the patient beats.

19. The method of claim 12, wherein advancing the tip of the medical device through the vasculature of the patient includes advancing the tip of the medical device through a right internal jugular vein, a right brachiocephalic vein, and into the SVC.

20. The method of claim 19, wherein the medical device is a central venous catheter (“CVC”).

21. The method of claim 12, wherein advancing the tip of the medical device through the vasculature of the patient includes advancing the tip of the medical device through a right basilic vein, a right axillary vein, a right subclavian vein, a right brachiocephalic vein, and into the SVC.

22. The method of claim 21, wherein the medical device is a peripherally inserted central catheter (“PICC”).
